(19) United States
(12) Patent Application Publication
(10) Pub. No.: US 2008/0143960 A1
(43) Pub. Date: Jun. 19, 2008

(54) Apparatus And Method For Simulating Vision Correction

(76) Inventor: Scott M. MacRae, Rochester, NY (US)

Correspondence Address: BLANK ROME LLP, 600 NEW HAMPSHIRE AVENUE, N.W., WASHINGTON, DC, 20037, US

(21) Appl. No.: 11/791,227

(22) PCT Filed: Nov. 22, 2005
(86) PCT No.: PCT/US2005/042368

§ 371 Date: Oct. 5, 2007

Related U.S. Application Data
(60) Provisional application No. 60/629,338, filed on Nov. 22, 2004.

(51) Int. Cl. A61B 3/04

(52) U.S. Cl. 351/230

(57) ABSTRACT

An ophthalmic diagnostic device is disclosed having a trial spectacle frame (100), phoropter (900), or other device adapted to be placed proximate a living eye of a patient, and having a set of trial lenses (300) adapted to be attached to the device. At least one of the trial lenses is adapted to correcting a higher order aberration associated with the living eye of the patient, including correcting a third, fourth and fifth higher order aberration or a combination of higher order aberrations as selected by the patient's healthcare practitioner.

### CROSS REFERENCE TO RELATED APPLICATIONS

This application is related to U.S. Provisional Patent Application Ser. No. 60/629,338, entitled “VISION DIAGNOSTIC DEVICE,” filed on Nov. 22, 2004, which is expressly incorporated by reference herein.

### BACKGROUND OF THE INVENTION

1. Field of Invention

The present invention relates to systems and methods for simulating vision correction. In particular, the present invention relates to a trial lens set for identifying or confirming aberrations of a living eye of a patient and simulating correction of higher order aberrations in the patient's eye.

2. Description of the Related Art

Higher order aberrations are refractive errors in the living eye, other than nearsightedness, farsightedness, and astigmatism, which cannot be corrected with currently-manufactured glasses or contact lenses. University of Rochester researchers were pioneers in the discovery and classification of higher order aberrations, which has led to many new and improved ophthalmic vision correction equipment, surgical and analytical methods, and diagnostic tools. For example, advanced refractive surgery techniques, such as wavefront-guided laser eye surgery, and improved laser assisted in situ keratomileusis (LASIK) techniques, have resulted directly from University of Rochester research. Similarly, some of the new and improved ophthalmic vision correction diagnostic tools involve identifying corneal wavefront aberrations and retinal image resolution. Devices and techniques continue to be developed as an understanding of higher order aberrations becomes more widely distributed among research and diagnostic health care providers.

U.S. Pat. Nos. 6,712,466, 6,840,619, and 6,942,339 disclose an eyeglass lens that was developed from this new understanding of higher order aberrations. The three patents disclose a lens manufacturing method whereby two lenses are used to sandwich a layer of variable index material, such as ultraviolet light-curable epoxy, between them to form a composite lens system. The UV-curable epoxy is cured to different indexes of refraction over the surface of the epoxy, which provides corrections for a patient's wavefront aberrations. The three patents also disclose a method for producing an eyeglass that corrects higher order aberrations, such as those that occur when retinal tissue is damaged due to glaucoma or macular degeneration. As part of the disclosed manufacturing method, the patents disclose the diagnostic step of imaging a patient's eye with a wavefront sensor in order to determine a wavefront prescription for the patient, but they do not mention other specific diagnostic methods.

The most widely used and well-established diagnostic methods used to measure performance of the ocular system are the psycho-physical methods that rely on subjective feedback from a patient. The oldest of those methods still used today by ophthalmologists and optometrists in an office or clinical setting is the phoropter or trial lens method. Typically, a trial lens is used to adjust the patient's refraction during testing before a final eyeglass or contact lens is selected or manufactured for the patient's daily use. Trial lenses can be placed in front of a patient's eye during examination by use of a trial frame, phoropter, or other device. Each of the psycho-physical diagnostic methods relies on trial-and-error to determine the required vision correction.

A traditional trial lens is shown in U.S. Pat. No. 3,947,186, which describes an assembly comprising a lens holder and a trial lens. Standard eyeglass trial lens sets, with convenient storage briefcases and trial frames for holding individual trial lenses, are sold commercially by, for example, Richmond Products of Albuquerque, N.Y., Reichert, Inc. of Depew, N.Y., and other ophthalmic equipment vendors. Traditionally, trial lens sets have included a plurality of individual lenses for diagnosing only astigmatism, myopia, and/or hyperopia, i.e., lower order aberrations. For example, a trial lens set may have several dozen individual spherical and cylindrical, concave and convex, trial lenses each with different refractive powers.

U.S. Patent Application Publication No. 2005/0124983 discloses a trial lens wheel having a plurality of traditional constant refractive index trial lenses with different refractive powers attached thereto that are used in the diagnosis and assessment of vision acuity in patients. U.S. Pat. No. 6,902,273 discloses a method of designing an ophthalmic lens using, as a starting point, a traditional trial lens.

Trial lenses are also being used, in combination with wavefront sensors, to diagnose higher order aberrations. U.S. Patent Application Publication No. 2005/0105048, for example, discloses an eye measurement system located at the office of an optometrist that is configured to measure a patient's vision parameters and to obtain measured optical aberration data. The application discloses that the eye measurement system may be a phoropter, autorefractor, or trial lens that can be used to produce eye measurement data that represents optical low and/or high order aberrations, but it does not mention the use of trial lenses having higher order optical characteristics.

Similarly, U.S. Patent Application Publication No. 2005/0105043 discloses a method of making corrective eyeglass lenses that involves obtaining vision parameters of a patient's eyes using, for example, a trial lens set, and then forming a lens that corrects at least one higher order aberration. The application, like the one described above, discloses that an eye measurement system can produce eye measurement data that represents optical low and/or higher order aberrations, but it does not mention the use of trial lenses having higher order optical characteristics.

In U.S. Patent Application Publication No. 2004/0057014, a method for determining a surface parameter of an ophthalmic correcting surface from a wavefront aberration measurement of an eye is disclosed, whereby the determination is done by obtaining a wavefront aberration measurement of a patient's eye at a known measurement plane location using a known measurement wavelength. The application discloses obtaining a wavefront aberration measurement that can be accomplished by making the measurement through a trial lens engaged with the patient's eye, but the application does not mention the use of trial lenses having higher order optical characteristics.

Trial lenses may also be in the form of contact lenses. For example, U.S. Patent Publication No. 2003/0107703 discloses a contact lens having a physical design parameter determined by an objective wavefront measurement of a plurality of in-situ trial contact lenses each having a different value of the physical design parameter for a given correcting power. U.S. Pat. No. 6,808,265 also discloses using trial contact lenses, as well as intraocular lenses.

As indicated above, trial lenses have been typically used to diagnose lower order aberration problems, i.e., those produced by cylindrical and spherical aberrations of a living eye, which in turn can be used to prescribe eyeglass lenses, contact lenses, and possibly other types of lenses. Trial lenses have also been used in conjunction with wavefront diagnostic systems to remove lower order aberrations in order to better focus reflected wavefront energy which provides for a better diagnosis of higher order aberration problems.

It should be apparent, therefore, that there exists a need for a trial lens set that incorporates optical properties for correcting higher order aberrations, not just traditional spherical and cylindrical lower order aberrations, and there exists a need for a trial eyeglass or spectacle frame, a phoropter, or other suitable diagnostic device for use with the trial lenses.

### SUMMARY AND OBJECTS OF THE INVENTION

Accordingly, it is a principal object of the present invention to provide a higher order aberration diagnostic device for diagnosing visual acuity problems in a living eye.

It is another object of the present invention to provide a trial spectacle lens, trial phoropter lens, trial contact lens, and/or other type of trial lens set having at least one trial lens adapted to correcting a higher order aberration problem of a living eye.

It is still another object of the present invention to provide a trial lens set having a plurality of trial lenses, wherein at least one of the plurality of trial lenses is adapted to correcting a higher order aberration, including at least third, fourth, and fifth higher order modes (however, up to tenth order or above would be contemplated).

It is another object of the present invention to provide a carrying case adapted to holding the trial lenses.

It is still another object of the present invention to provide a trial lens wheel adapted to holding a plurality of individual trial lenses.

It is another object of the present invention to provide a complete ophthalmic diagnostic device having a trial spectacle frame adapted to be worn proximate a living eye of a patient, a plurality of trial lenses adapted to be attached to the trial frame, and a carrying case for holding the trial frame and the plurality of trial lenses, such that at least one of the plurality of trial lenses is adapted to correcting a higher order aberration associated with the living eye of the patient, including third, fourth, and/or fifth higher order aberrations.

It is another object of the present invention to provide a phoropter for diagnosing visual acuity problems having a phoropter frame adapted to being suspended proximate a living eye of a patient, the frame having at least one through hole; at least one trial lens frame positioned within the phoropter frame; and a plurality of trial lenses adapted to being attached to the trial lens frame, such that the trial lens frame is moveable relative to the phoropter frame so that at least one of the trial lenses is positioned proximate the through hole, and wherein at least one of the plurality of trial lenses is adapted to correcting a higher order aberration associated with the living eye of the patient.

It is still another object of the present invention to diagnose and simulate correction of visual acuity problems associated with astigmatism and myopia together with higher order aberrations.

Briefly described, these and other objects and features of the present invention are accomplished, as embodied and fully described herein, by a higher order aberration diagnostic device that is adapted to identify aberrations and simulate the correction of those aberrations. The device includes a trial lens set having a first trial lens, wherein the first trial lens is adapted to being positioned proximate a living eye for identifying a higher order aberration associated with the eye. The trial lens set may further have a second trial lens, wherein the second trial lens is adapted to being positioned proximate the eye for identifying a second higher order aberration or, in combination with the first trial lens, for identifying the higher order aberration. The trial lens may be a spectacle lens, contact lens, or other lens type.

The objects and features of the present invention are also accomplished, as embodied and fully described herein, by a trial lens set having a plurality of trial lenses, wherein at least one of the plurality of trial lenses is adapted to being positioned proximate the eye for identifying and simulating the correction of a higher order aberration, which could be a second, third, fourth, or fifth mode aberration. The invention may also include a carrying case adapted to holding the plurality of trial lenses. The plurality of trial lenses may be spectacle, contact, or some other type of lens.

The objects and features of the present invention are also accomplished, as embodied and fully described herein, by an ophthalmic diagnostic device having a trial spectacle frame adapted to be worn proximate a living eye of a patient, a plurality of trial lenses adapted to be attached to the trial frame, and a carrying case for holding the trial frame and the plurality of trial lenses. In that embodiment, at least one of the plurality of trial lenses is used to identify and simulate the correction of a higher order aberration associated with the living eye of the patient, including correcting a third, fourth and fifth higher order aberration or a combination of higher order aberrations as selected by the practitioner.

The objects and features of the present invention are accomplished, as embodied and fully described herein, by an ophthalmic device for diagnosing visual acuity problems, the device having a phoropter frame adapted to being placed proximate a living eye of a patient, the frame having at least one through hole; at least one trial lens frame positioned within the phoropter frame; and a plurality of trial lenses adapted to being attached to the at least one trial lens frame. The at least one trial lens frame is moveable relative to the phoropter frame such that at least one of the plurality of trial lenses is positioned proximate the at least one through hole, and at least one of the plurality of trial lenses is adapted to identifying and simulating the correction of a higher order aberration associated with the living eye of the patient. In that embodiment, at least one of the plurality of trial lenses is adapted to identifying astigmatism and at least one of the plurality of trial lenses is adapted to identifying myopia. The at least one trial lens frame may be a wheel that is rotatably moveable relative to the phoropter frame and is adapted to identify and simulate the correction of different higher order aberrations as described by Zernike, Fourier, or any other mathematical method of describing wavefront error.

Finally, the object and features of the present invention are accomplished, as embodied and fully described herein, by a method for using trial lenses to identify higher order aberrations in the living eye of a patient and simulating the correction of those aberrations prior to prescribing a lens for use by the patient.

With those and other objects, features, and advantages of the invention that may become hereinafter apparent, the nature of the invention may be more clearly understood by reference to the following detailed description of the invention, the appended claims and to the several drawings attached herein.

### BRIEF DESCRIPTION OF THE DRAWINGS

FIG. 1 is a drawing of a perspective view of a prior art trial lens spectacle frame;

FIG. 2a is a drawing of a partially exploded perspective view of a trial lens set carrying case according to the present invention;

FIG. 2b is a drawing of a trial lens set according to the present invention;

FIG. 3 is a drawing of a perspective view of a trial lens set according to the present invention;

FIG. 4 is a perspective drawing of Zernike polynomials for the second through fifth order aberrations;

FIG. 5 is a drawing of the point spread functions associated with the Zernike polynomials shown in FIG. 4;

FIG. 6 is a drawing of a plan view of one embodiment of the trial lenses in the trial lens set shown in FIG. 3;

FIG. 7 is a drawing of a perspective view of some of the trial lenses in the trial lens set shown in FIG. 3;

FIG. 8 is a drawing of a perspective view of some lower and higher order correction trial lenses in the trial lens set shown in FIG. 3;

FIG. 9 is a drawing of a perspective view of a phoropter incorporating higher order correction trial lenses according to the present invention; and

FIG. 10 is a process flow diagram according to one aspect of the present invention.

### DETAILED DESCRIPTION OF THE PREFERRED EMBODIMENT

Several preferred embodiments of the invention are described for illustrative purposes, it being understood that the invention may be embodied in other forms not specifically shown in the drawings.

Tuning first to FIG. 1, shown therein is a perspective view of a prior art trial lens spectacle-type frame 100. A trial lens frame, also known as a simple phoropter, is used to hold trial lenses proximate a living eye so a doctor can simulate a patient's corrected vision in a clinical setting, such as at a doctors office. Often, patients suffering from myopia (nearsightedness, short-sightedness), hypermetropia (farsightedness, long-sightedness), astigmatism, or higher order aberrations may not fully appreciate his or her corrected vision by simply sitting behind a suspended phoropter and looking at the letters and numbers on an spot-lit acuity chart. By wearing the trial frame 100, the patient can walk around the office or outside, read a book, look at distant and near objects, and simulate other activities wearing a pair of prescription lenses. The patient can then appreciate what the final correction of both lower order and higher order aberrations will be like after treatment with laser refractive surgery, contact lenses, or spectacles. A doctor may also then be able to better diagnose aberrations after the patient, wearing the trial lens frame 100, reports any subjective image quality problems that may not have been diagnosed earlier.

The trial lens frame 100 includes lens subframes 102, 104, each having rim portions 106, 108, respectively. The lens subframes 102, 104 may each be separated into at least two portions to facilitate attaching trial lenses 150, 152 to the rim portions 106, 108. For example, the two lens subframes portions may open by way of devices 110, 112, which may be hinges (not shown), a track (not shown), or other suitable mechanism.

Additional lens subframes (not shown) could also be used disposed directly in front of or behind the lens subframes 102, 104 to hold additional lenses (not shown) so that a plurality of lenses may be positioned in front of a patient's eyes. Preferably, up to 4 lens subframes on each half of the trial frame 100 would be used, at least one of which could be used to hold a second order aberration lens (described in more detail below). The lens subframes and trial lenses should be thin so that the total combined thickness of the lenses as well as the vertex distance (i.e., the distance from the eye to the lens) is as small as possible.

The trial lenses themselves may have a device for attaching the lens to the trial lens frame 100 without the need for the lens subframes 102, 104. The lens subframes 102, 104, or the trial lenses 150, 152 (and the additional lens subframes and trial lenses, if used) may each be rotatable 360 degrees so that the trial lenses 150, 152 may each be rotated independently from each other to match the rotational degree of aberration of the patient's eyes. Grip portions 114, 116 on each of the lens subframes may be included to facilitate rotating the lens subframes 102, 104.

Also, one of ordinary skill in the art will appreciate that a trial lens frame may be a hand-held device and may have only one trial lens attached thereto for positioning in front of only one eye of a patient.

Although the trial lens frame 100 by itself is described above as prior art, once the trial lenses of the present invention are attached to the trial lens frame 100, the combination of trial lenses and trial lens frame 100 is contemplated to be within the scope of the present invention.

One of ordinary skill in the art will also appreciate that the trial lens frame 100 may be in the shape of a monocular, binocular, camera, telescope, safety glasses, sunglasses, or any other device incorporating optical lenses, to better simulate for the patient how effective the device may work when the patient's vision is corrected using actual lenses.

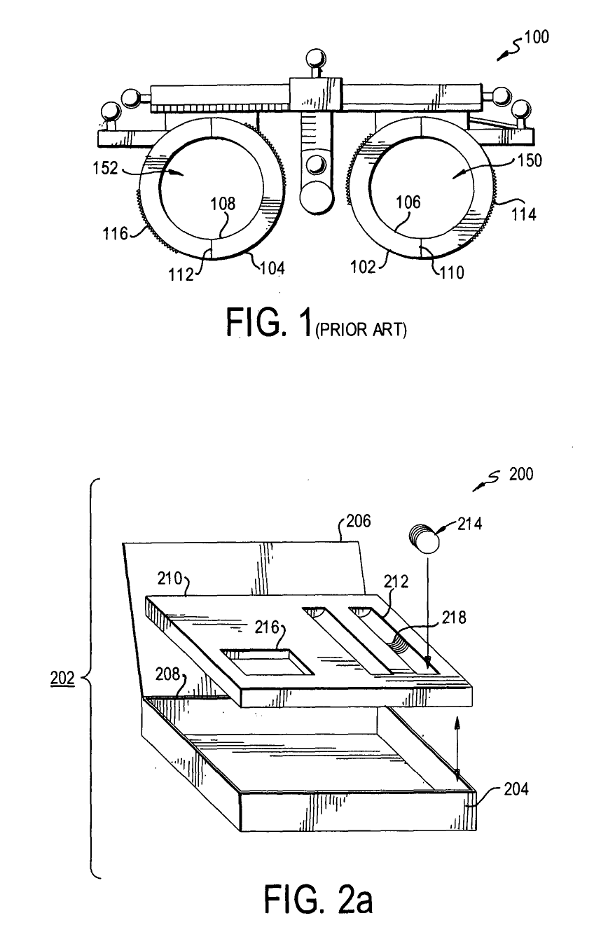

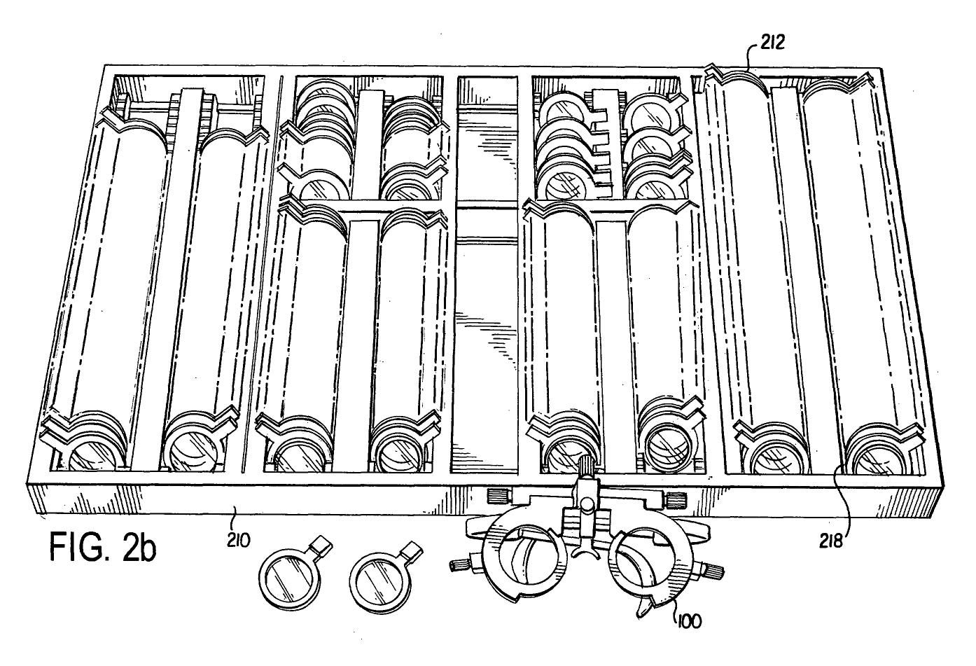

FIG. 2a is a partially exploded perspective view of a trial lens set carrying case 200 according to the present invention. The trial lens set carrying case 200 preferably includes a case 202, which in the embodiment shown in FIG. 2a is simply a rectangular box having a base portion 204 and a lid portion 206. The lid portion 206 may or may not be attached to the base portion 204. If it is attached, the lid portion 206 may be attached using a hinge 208 or any other suitable device. The case 202 may have other standard features such as a clasp, lock, and handle (not shown).

The base 204 is adaptable to holding one or more removable trays. In FIG. 2a, a single removable tray 210 is shown for illustrative purposes. The tray 210 may include one or more compartments. For example, the compartment 212 may include a plurality of slots 218 for securing a plurality of trial lenses 214 in an upright manner (as best seen in FIG. 2b), thereby preventing the lenses from touching each other during storage and transport. tray or within a compartment of another tray. The tray 210 and case 202 should be made of durable and shock-absorbing material to facilitate transport of the trial lens set carrying case 200. Trial lens trays like that shown in FIG. 2b are commercially available in a variety of sizes and compartment configurations from companies like Lombart, Zeiss, Bausch & Lomb, and others.

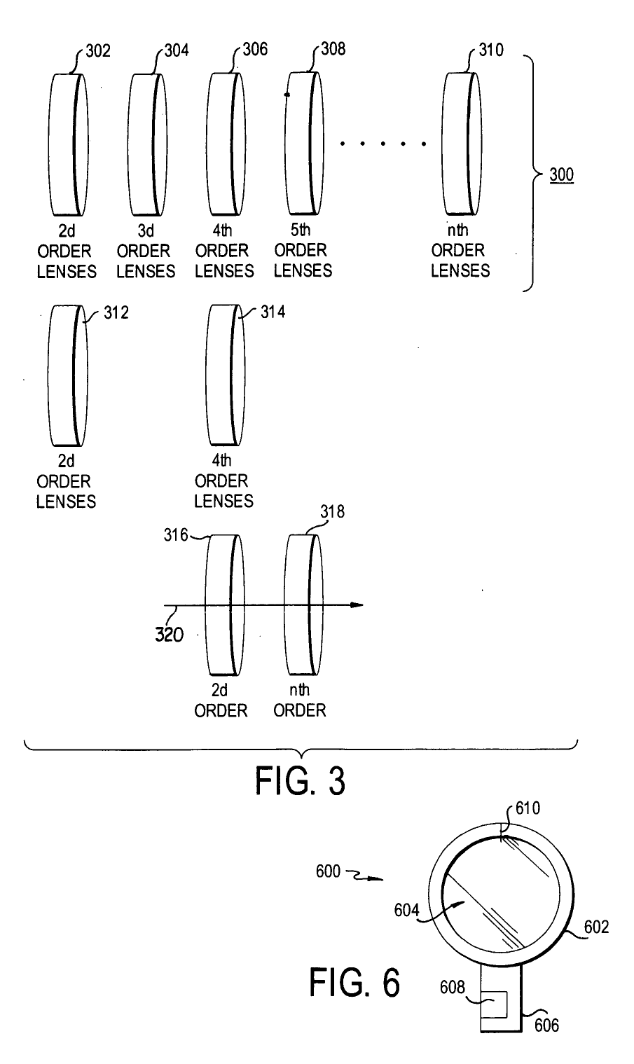

Turning now to FIG. 3, shown therein is a perspective view of a trial lens set 300 according to the present invention. The trial lens set 300 may consist of a plurality of spectacle, contact, or a mix of those or other types of lenses. The invention is best suited for spectacle or contact lenses, but it may also be used in connection with developing technologies, such as adjustable intraocular lenses (i.e., post-surgical, external stimulus-induced polymerization intraocular lenses, such as those disclosed in U.S. Patent Application Publication No. 2003/0151825), despite the difficulties and feasibility associated with using trial intraocular lenses.

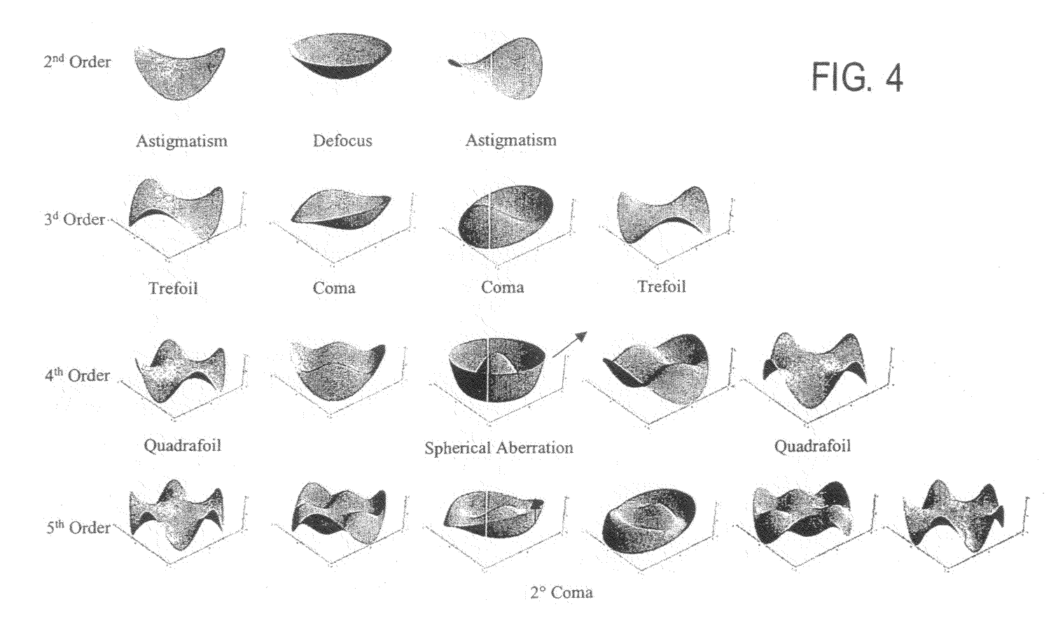

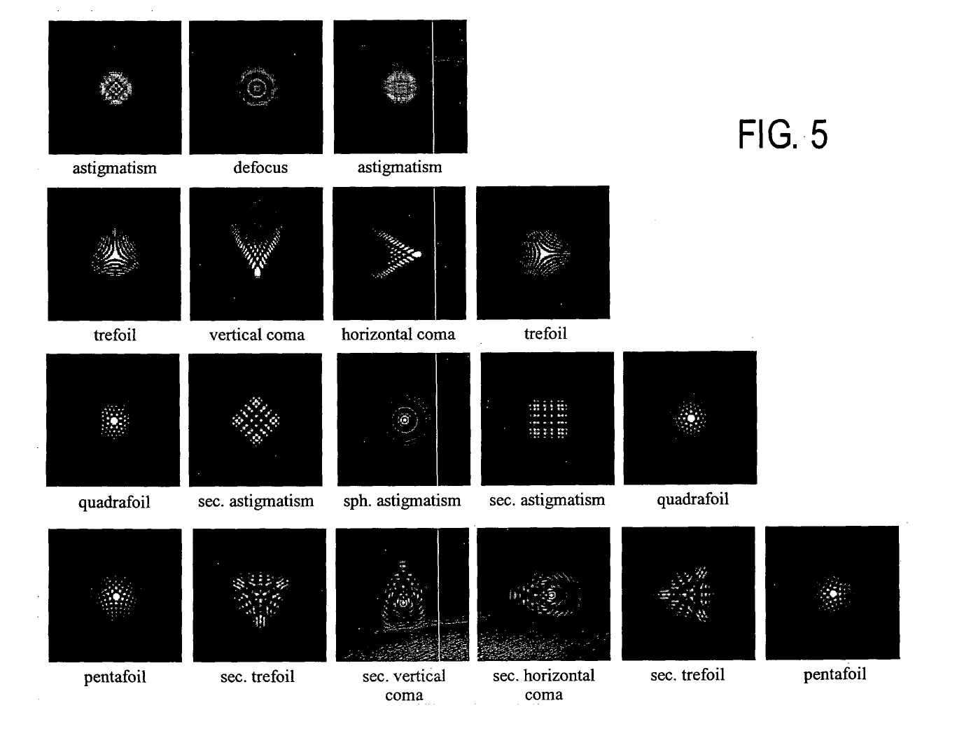

The trial lenses may be grouped and labeled according to Zernike modes or other types of aberrations, or other types of aberrations descriptions (i.e., Fourier). As illustrated in FIGS. 4 and 5, Zernike modes refer to polynomials, which are mathematical descriptions of ocular aberrations in the optical properties of a living eye. The polynomials are measured with wavefront technology known in the art. The respective point spread functions are shown in FIG. 5, which demonstrates the image of a point source of light (e.g., a star or a head light) when the eye has an isolated aberration noted in FIG. 4.

Each Zernike mode is associated with a polynomial of a certain three-dimensional shape that describes the wavefront error of a living eye. For example, the second order Zernike terms represent the traditional aberrations defocus (i.e., spherical correction related to myopia and hyperopia) and astigmatism. The third order Zernike terms, discussed further below, are coma (i.e., a wavefront shape having a twofold peak and valley symmetry as best seen in FIG. 5) and trefoil (i.e., a wavefront shape having a threefold symmetry). The fourth order Zernike terms include spherical aberration and four other terms. Although the Zernike polynomials are an infinite set, ophthalmologists generally limit discussion to the first twelve orders, and preferably the first five orders, since the magnitude of aberrations observed in normal clinical settings are typically more pronounced in the lower orders.

For example, the trial lens set 300 in FIG. 3 may include one or more second or lower order aberrations trial lenses 302. Those second or lower order aberrations trial lenses 302 may include one or more individual trial lenses 312 for correcting various degrees of astigmatism and one or more individual lenses 314 for correcting various degrees of myopia or hyperopia, among other lenses.

The trial lens set 300 also includes one or more higher order aberrations lenses, including one or more third order aberrations trial lenses 304, one or more fourth order aberrations trial lenses 306, one or more fifth order aberrations trial lenses 308, etc., up to one or more nth order aberrations trial lenses 310. The nth order Zernike mode may be described as a tenth order aberration, for example. Thus, a complete trial lens set, according to one embodiment of the invention, may consist of different second through tenth order trial lenses.

As discussed above, the purpose of the trial lenses is to identify visual acuity problems diagnostically by allowing simulation of corrected vision with higher order aberration. This can be accomplished in several ways. One way is to measure the aberrations in a patient's eye using, for example, wavefront sensing techniques. One could then adjust the higher order aberrations, depending on the patient's feedback, similar to a conventional subjective refraction process. Thus, the ophthalmologist or optometrist may select one or more trial lenses from the trial lens set 300 based on wavefront refraction measurements, attach those lenses to the trial lens frame 100, and allow the patient to wear the trial lens frame 100 so the patient may have a sense of what his or her corrected vision will be like. Alternatively, the ophthalmologist or optometrist may select one or more trial contact lenses from the trial lens set 300, apply the trial contact lenses to the eyes of the patient, and allow the patient to wear the trial lenses. Further still, the ophthalmologist or optometrist may select one pair of trial contact lens and one pair of trial spectacle lenses from the trial lens set 300, apply the trial contact lenses to the eyes of the patient and fit the trial spectacle lenses to a lens frame (making an appropriate vertex adjustment as needed) and allow the patient to wear the combination of trial lenses. Different trial lenses may then be used, depending on feedback from the patient.

Also as shown in FIG. 3, one of ordinary skill will appreciate that a higher order aberration trial lens 318, for example, may be combined with a lower or second order trial lenses 316 (i.e., placed in series relative to an optical path 320) in the trial lens frame 100 to correct sphere, cylinder, and cylinder rotation (axis) and better simulate the aberrations to be corrected by ocular devices, contact lenses, and other types of lenses. Preferably, a practitioner may wish to combine four lenses in series to correct a patient's vision problems by simultaneously using at least one second order (as needed) and several different higher order trial lenses.

FIG. 6 is a plan view of a trial lens 600 from the trial lens set 300 shown in FIG. 3. The trial lens 600 may include a lens frame 602, lens 604, handle 606, and label 608. A protective sleeve (not shown) may be used to store the trial lens 400 in the carrying case 202 (FIG. 2). The label 608 may contain indicia describing the physical properties of the lens 604. The handle 606 may be used to hold the trial lens 400 while it is being inserted in the trial lens frame 100 (FIG. 1). The indicia 610 is used to identify the axis or rotational angle of the lens 604. The indicia 610 may be a printed mark, a physically raised portion, or some other means for indicating the axis or rotational angle of the lens 604. The indicia 610 may be located on either the lens frame 602, the lens 604, or on both.

Where the trial lens 600 is a contact lens, no handle 606 would be required (or desired). Instead, a peripheral edge of the trial lens 600 may include indicia (not shown) describing the physical properties of the contact lens 604. If the lens 604 is a contact lens, it may be stored individually in a sealable bottle (not shown) with an appropriate label adhered to the bottle indicating the contents therein. For a contact lens, the indicia 610 would be located on the lens 604 itself.

One of ordinary skill in the art will understand that the contact trial lens would need to be stabilized so it does not rotate excessively. That can be accomplished using a prism ballast system or some other method which is known in the art to allow the contact lens to settle at the proper axis on the patient's cornea. That is essentially what is done with patients with astigmatism where they receive a custom fabricated toric soft or hard contact lens with a pre-established amount of astigmatism which will correct at a particular axis when it settles and stabilizes on the cornea.

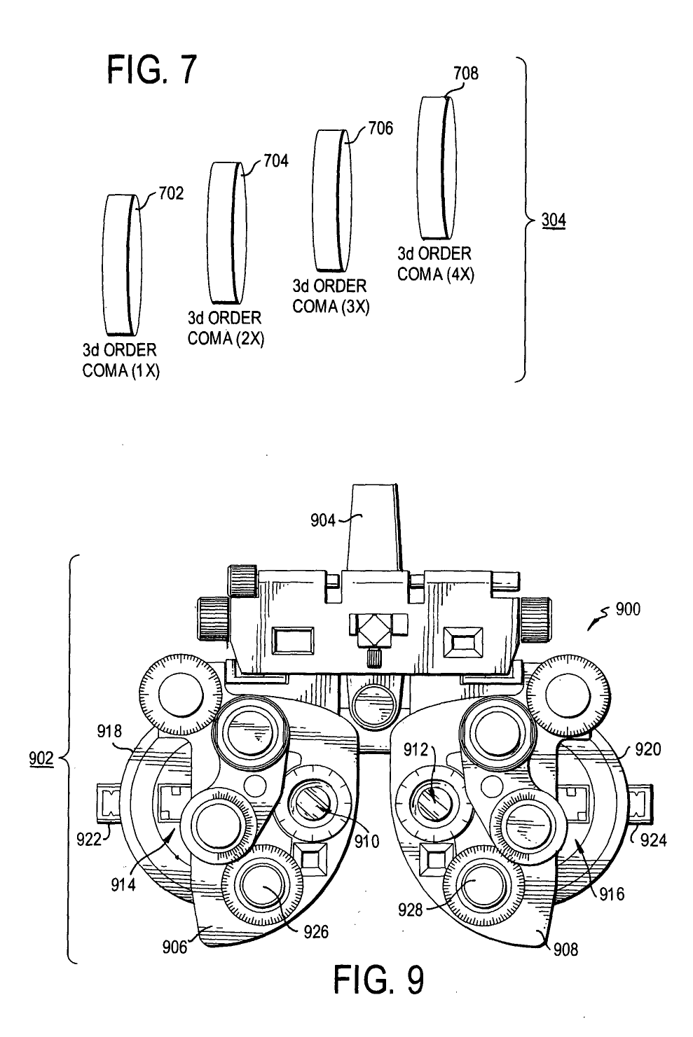

FIG. 7 is a perspective view of some of the trial lenses in the trial lens set 300 shown in FIG. 3.

According to the present invention, there may be multiple trial lenses for each higher order aberration. Thus, for example, as shown in FIG. 7, the third order trial lenses 304 (FIG. 3) may consist of trial lens 702 (third order, 1× coma), trial lens 704 (third order, 2× coma), trial lens 706 (third order, 3× coma), and trial lens 708 (third order, 4× coma), where the indicia 1×, 2×, etc., refer to the magnitude or degree of aberration relative to a baseline.

Different magnitudes or degrees of aberration may be included for each trial lens type. For example, the 1×, 2×, 3×, etc. magnitudes may be measured relative to a population manifesting typical expected or normal higher order aberrations. Additionally, the 1×, 2×, 3×, etc. magnitudes may be measured relative to a population manifesting greater magnitudes of higher order aberrations (such as keratoconuous and post corneal transplant patients). Thus, the magnitudes 1× in each group might be completely different.

Moreover, the magnitudes or degrees of aberration do not have to be simple whole number increments. Rather, the magnitudes or degrees of aberration for the individual lenses of a particular type of higher order trial lens may be 0.5×, 1×, 1.5×, or n×, where n can be any real number. Thus, for example, the trial lens set 300 may have one each 0.25×, 0.5×, 1×, 2×, and 4× third order coma trial lens. Obviously, combinations of those lenses can also be used to obtain a magnitude not provided by a single lens. For example, a 2× lens combined with a 0.5× lens can provide a total magnitude of 2.5×.

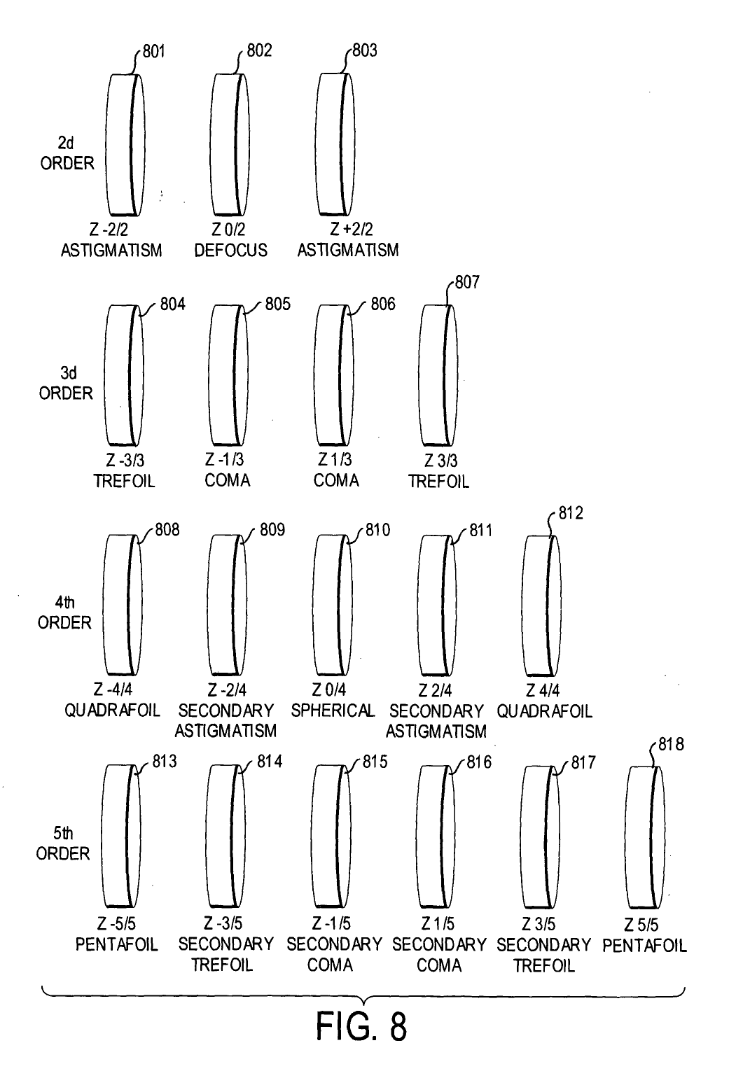

FIG. 8 is a perspective view of some lower and higher order correction trial lenses in the trial lens set 300 shown in FIG. 3. There may be two groups of second order trial lenses 801, 802, corresponding to astigmatism and defocus, respectively. There may be two third order trial lenses 804, 805, corresponding to trefoil and coma aberrations, respectively. There may be three fourth order trial lenses 808, 809, and 810, corresponding to quadrafoil, secondary astigmatism and spherical aberrations, respectively. Finally, there may also be three fifth order trial lenses 813, 814, and 815, corresponding to pentafoil, secondary trefoil, and secondary coma aberrations, respectively. (Not shown are sixth order trial lenses, seventh order trial lenses, etc., up to tenth order trial lenses, where the lenses are grouped according to Zernike modes). The preferred trial lens set 300 will have at least the third order trefoil aberration trial lens 804, at least the third order coma aberration trial lens 805, and at least the fourth order spherical aberration trial lens 810, because those aberrations are the most common aberrations observed in the population of patient's suffering from higher order aberrations.

One of ordinary skill in the art will appreciate that there are other ways of describing higher order aberrations, including Fourier analysis and other methods, and thus other ways of grouping the trial lenses in the trial lens set 300.

As shown in FIG. 5, the point spread functions of some of the higher order aberrations are symmetrical about an axis, but they are rotated by a finite amount. For example, the third order vertical coma and horizontal coma point spread functions are identical, just rotated about the plane of the page by 90 degrees. The third order trefoil point spread functions are also identical, just rotated about the plane of the page by 60 degrees. Thus, if a patient suffers from a third order coma aberration disposed with its rotational degree of aberration axis at, say, 60 degrees (measured relative to the horizontal axis of the page in FIG. 5) an ophthalmologist or optometrist may simply select a coma trial lens, attach it to the trial frame, and rotate the lens until it aligns with or matches the patient's 60 degree third order coma aberration. The ophthalmologist or optometrist would then select different magnitudes (i.e., 0.25×, 0.5×, 1×, 2×, or n×) and combinations of magnitudes of the third order coma trial lens until the patient's visual acuity is improved. A wavefront sensor system may be used by the practitioner as a guide for narrowing the direction and magnitude of the aberration to speed the process of selecting and orienting the proper trial lens or combination of trial lenses. A higher order refraction can also be conducted with a phoropter or with retinoscopy to optimize the optical correction as well.

Accordingly, while the preferred trial lens set 300 will include a set of four third order coma trial lenses 805 (with each individual third order coma trial lens corresponding to a different magnitude or degree of aberration), the trial lens set may also include an additional set of four third order trial lenses 806, especially where the trial lens frame 100 does not allow the trial lens to be rotated 360 degrees (which is often the case where the trial lenses have a handle 606 as shown in FIG. 6, because the handles may bump into the trial lens frame 100 or the patient's nose).

Similarly, the preferred trial lens set 300 may have an additional set of second order trefoil trial lenses 807 for the same or other reasons. Moreover, the trial lest set 300 may have an additional set of secondary astigmatism trial lenses 811 and quadrafoil trial lenses 812, as well as sets of fifth order secondary coma trial lenses 816, secondary trefoil trial lenses 817, and pentafoil trial lenses 818.

FIG. 9 is a perspective view of a standard boom-supported phoropter 900 adapted to incorporating higher order correction trial lenses according to the present invention. The traditional phoropter 900 includes a frame 902 attached to a boom 904. The boom 904 is a reticulated and adjustable arm that supports the phoropter 900 in front of a patient's face during a diagnostic visual acuity exam. The particular phoropter 900 shown in FIG. 9 is similar to one made by Riechert and sold commercially, but any traditional phoropter with slight structural modifications may be used.

The phoropter frame 902 includes first and second spaced apart sides 906, 908. Each side has a through hole 910, 912, that a patient can see through to view an acuity chart. Interiorly of the phoropter frame 902 are first and second trial lens frames 914, 916, each holding a plurality of trial lenses (not shown). Preferably, the first and second trial lens frames 914, 916 are in the shape of a wheel that is rotatably mounted inside the phoropter frame 902. The trial lens frames are interconnected to the selector wheel 918, 920, which allow a user to select a trial lens on the trial lens frames 914, 916 such that the trial lenses may be positioned in the through holes 910, 912. Adjusters 922, 924 are used to check the vertex distance. Selector knobs 926, 928 are interconnected with trial lens frames 914, 916 and are used to rotate the individual trial lenses to a proper axis/angle when the trial lenses are positioned in the through holes 910, 912. They are also used to select the appropriate magnitude for the particular lenses. An opening in the phoropter frame 902 is provided (not shown), so that the trial lenses on the trial lens frames 914, 916 may be replaced.

In order to accommodate a large number of trial lenses, each side 906, 908 of the phoropter frame 902 may house several trial lens frames. Each trial lens frame is mounted on the same axis so that multiple trial lenses may be positioned in the through holes 910, 912. Preferably, up to four (or more) trial lenses, each on a different trial lens frame, may be positioned in series in the through holes 910, 912. Each trial lens frame will be interconnected to the selector wheel 918, 920 (or to separate selector wheels, one associated with each trial lens frame), and each individual trial lens will be interconnected to the selector knobs 922, 924 (or to separate selector knobs, one associated with each trial lens frame) so that each trial lens can be rotated 360 degrees in the through holes 910, 912.

One of ordinary skill in the art will appreciate that the wheel trial lens frames 914, 916 may be replaced with other devices and achieve the same functionality as that of the described embodiment. For example, an autorefractor may be used. Also, a slidable cartridge having the trial lenses mounted side-by-side between two spaced apart rails could be used.

To enhance the capabilities of the phoropter 900, the device may be operatively connected to a wavefront sensor via a network or cable (not shown), using computers to automatically dial the appropriate combination of trial lenses in the phoropter 900. That automation reduces the time it takes to select the trial lenses, move them into position in the through holes 910, 912, rotate the higher order lenses to the proper angle/axis, and select an appropriate magnitude. The phoropter 900 may also have an interface for receiving wavefront data electronically stored on a portable electronic data storage device.

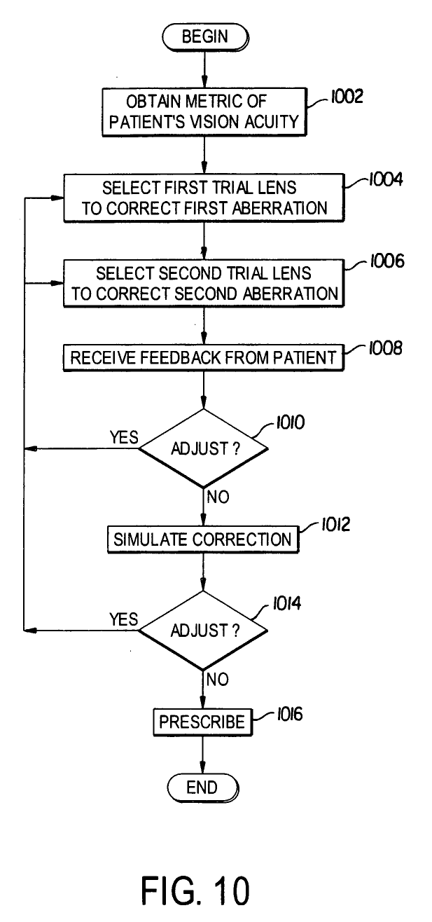

FIG. 10 is a process flow diagram according to one aspect of the present invention. In step 1002, a healthcare practitioner, such as an ophthalmologist or optometrist, obtains a metric of a patient's visual acuity through subjective or objective information or by taking measurements using an instrument. For example, the patient may have prescription eyeglasses that can be evaluated to estimate a refractive power metric. The patient may have medical records containing previous prescription information. Or, a wavefront sensor may be used to estimate aberrations in the patient's eyes.

In step 1004, the practitioner selects a first trial lens to identify an aberration associated with the patient's eyes. Based on the subjective feedback from the patient, or based on the feedback plus objective metric information, in step 1006, the practitioner selects a second trial lens to identify an aberration associated with the patient's eyes. In step 1008, the practitioner receives patient feedback on the combination of the first and second trial lenses. This is an iterative process, as shown in decision step 1010, that involves selecting trial lenses until the combination of patient feedback alone or in combination with objective metric information results in the selection of optimal corrective lenses for the patient.

In step 1012, the patient uses the optimal trial lenses to simulate his or her corrected vision. In step 1014, the selection of trial lenses may need to be adjusted based on feedback and/or other information. Finally, in step 1016, the practitioner prescribes corrective lenses, a custom LASIK procedure, contact lenses, and the like, for the patient.

Although certain presently preferred embodiments of the disclosed invention have been specifically described herein, it will be apparent to those skilled in the art to which the invention pertains that variations and modifications of the various embodiments shown and described herein may be made without departing from the spirit and scope of the invention. For example, the system can also be used with retinoscopy to neutralize the lower order and higher order aberrations. Accordingly, it is intended that the invention be limited only to the extent required by the appended claims and the applicable rules of law.

I claim:

1. A trial lens set comprising:
a first trial lens adapted to be positioned proximate a living eye for identifying a higher order aberration in the eye.

2. The trial lens set according to claim 1, further comprising:
a second trial lens, wherein the second trial lens is adapted to be positioned proximate the eye for identifying a second higher order aberration in the eye.

3. The trial lens set according to claim 1, further comprising:
a second trial lens adapted to be positioned proximate the eye for identifying, in combination with the first trial lens, a higher order aberration in the eye as well as identifying one of a spherical and cylindrical aberration problem in the eye.

4. The trial lens set according to claim 1, wherein the first trial lens is adapted to simulate the correction of the higher order aberration associated with the eye.

5. The trial lens set according to claim 1, wherein the first trial lens is a spectacle lens.

6. The trial lens set according to claim 1, wherein the first trial lens is a contact lens.

7. The trial lens set according to claim 1, wherein the first trial lens is rotatable about an axis.

8. A trial lens set for identifying higher order aberrations and simulating correcting the aberrations, the trial set comprising:
a plurality of trial lenses,
wherein at least one of the plurality of trial lenses is adapted to be positioned proximate the eye for identifying a higher order aberration in the eye, and wherein at least one of the plurality of trial lenses is adapted to be positioned proximate the eye for identifying a spherical or cylindrical aberration in the eye.

9. The trial lens set according to claim 8, wherein the higher order aberration is one of a second, third, fourth, and fifth mode.

10. The trial lens set according to claim 8, further comprising a carrying case adapted to hold the plurality of trial lenses.

11. The trial lens set according to claim 8, wherein each of the plurality of trial lenses is one of a spectacle and contact lens.

12. The trial lens set according to claim 8, wherein each of the plurality of trial lenses is rotatable about an axis.

13. The trial lens set according to claim 8, wherein when the trial lenses are proximate the eye, the trial lenses simulate the correction of the aberrations.

14. The trial lens set according to claim 8, wherein the at least one of the plurality of higher order trial lenses is used with an autorefractor.

15. An ophthalmic diagnostic device comprising:
a trial spectacle frame adapted to be worn proximate a living eye of a patient;
a plurality of trial lenses adapted to be attached to the trial frame; and
a carrying case for holding the trial frame and the plurality of trial lenses,
wherein at least one of the plurality of trial lenses is adapted to identify a higher order aberration associated with the patient's eye.

16. The ophthalmic diagnostic device according to claim 15, wherein the plurality of trial lenses includes at least one trial lens adapted to identify a combination of third order aberrations.

17. The ophthalmic diagnostic device according to claim 16, wherein the plurality of trial lenses includes at least one trial lens adapted to identify a fourth higher order aberration and at least one trial lens adapted to identify a fifth higher order aberration.

18. The ophthalmic diagnostic device according to claim 15, wherein the plurality of trial lenses is adapted to rotate about an axis relative to the trial spectacle frame.

19. An ophthalmic device for identifying visual acuity problems comprising:
a phoropter frame adapted to be positioned proximate a living eye of a patient, the frame having at least one through hole;
at least one trial lens frame adapted to be removably attached to the phoropter frame; and
a plurality of trial lenses adapted to be removably attached to the at least one trial lens frame,
wherein the at least one trial lens frame is moveable relative to the phoropter frame such that at least one of the plurality of trial lenses is positioned proximate the at least one through hole, and
wherein at least one of the plurality of trial lenses is adapted to identify a higher order aberration associated with the living eye of the patient.

20. The ophthalmic diagnostic device according to claim 19,
wherein at least one of the plurality of trial lenses is adapted to identify one of astigmatism, power, and axis, and
wherein at least one of the plurality of trial lenses is adapted to identify one of myopia and hyperopia.

21. The ophthalmic diagnostic device according to claim 19, wherein the at least one trial lens frame is a wheel and wherein the wheel is rotatably adjustable relative to the phoropter frame.

22. The ophthalmic diagnostic device according to claim 19, further comprising means for rotating about an axis the at least one of the plurality of trial lenses when it is positioned proximate the at least one through hole.

23. The ophthalmic diagnostic device according to claim 19, wherein the at least one trial lens frame is a cartridge and wherein the cartridge is slidably adjustable relative to the phoropter frame.

24. The ophthalmic diagnostic device according to claim 19, further comprising means for receiving wavefront aberration information about the patient's eye in order to pre-select one of the plurality of trial lenses.

25. The ophthalmic diagnostic device according to claim 19, wherein the phoropter is used to simulate the correction of the aberration.

26. A method for identifying aberrations in a living eye and simulating the correction of the aberrations comprising the steps of:
selecting, from a trial lens set having both lower and higher order aberrations trial lenses, a first trial lens having an optical characteristic of a lower order aberration;
positioning the first trial lens proximate the eye;
selecting from the trial lens set a second trial lens having an optical characteristic of a higher order aberration;
positioning the second trial lens proximate the first trial lens;
iteratively selecting from the trial lens set a third trial lens to replace either the first or second trial lenses until the aberrations of the eye are identified; and
generating a prescription for a vision correcting lens, the prescription comprising information about the identified aberrations.

27. The method according to claim 26, wherein the optical axes of the first, second, and third trial lenses are aligned with the optical axis of the eye.

28. The method according to claim 26, wherein the trial lens are one of a spectacle lens and contact lens.

29. The method according to claim 26, further comprising the steps of:
after positioning the first trial lens proximate the eye, rotating the first trial lens about its optical axis; and
after positioning the second trial lens proximate the eye, rotating the second trial lens about its optical axis.

30. The method according to claim 26, further comprising the step of:
manufacturing the correcting lens based on the prescription information.

31. The method according to claim 26, further comprising the step of:
providing a means for simulating the correction of the aberrations prior to generating the prescription for the vision correcting lens.

32. A trial lens set for identifying lower order and higher order aberrations in a living eye, the trial set comprising:
a plurality of trial lenses,
wherein at least two of the plurality of trial lenses are adapted to be positioned proximate each other such that their optical axes are substantially aligned with the optical axis of the eye for identifying the low and high aberrations in the eye.

33. The trial lens set according to claim 32, wherein each of the plurality of trial lenses is one of a spectacle and contact lens.

34. The trial lens set according to claim 32, wherein each of the plurality of trial lenses is rotatable about its optical axis.
